(19) United States
(12) Patent Application Publication
(10) Pub. No.: US 2023/0226246 A1
(43) Pub. Date: Jul. 20, 2023

(54) MODULAR UV STERILIZATION SYSTEM FOR AIR PURIFICATION

(71) Applicant: Epic Universal Technologies, LLC, Hickory, NC (US)

(72) Inventor: George Erik McMillan, Hickory, NC (US)

(21) Appl. No.: 18/097,991

(22) Filed: Jan. 17, 2023

Related U.S. Application Data
(60) Provisional application No. 63/299,475, filed on Jan. 14, 2022.

(51) Int. Cl. A61L 9/18; F24F 8/22

(52) U.S. Cl. CPC A61L 9/18; F24F 8/22; F24F 2120/10

(57) ABSTRACT

A UV sterilization system is provided. In some embodiments, the UV sterilization system includes a first UV LED device having a LED circuit and LED emitters. The UV sterilization system can include a sensor electrically coupled to the first UV LED device, the sensor configured to interrupt the operation of the first UV LED device upon detecting movement within an air conditioning system. The UV sterilization system can include a power module electrically coupled to the sensor, where the power module is electrically coupled to a power supply of the air conditioning system. In some embodiments, the air conditioning system can include a packaged terminal air conditioner (PTAC) unit or a split-air air-conditioner system, and the UV sterilization system can be a part of the PTAC unit or split-air air-conditioning system. In some embodiments, the sensor includes a motion sensor and/or an occupancy sensor.

### CROSS-REFERENCE TO RELATED APPLICATION

This application claims the benefit of U.S. Provisional Patent Application No. 63/299,475, titled “PTAC/SPLIT-AIR/SMALL-FORM FACTOR AIR CONDITIONER MODULAR UV STERILIZATION SYSTEM,” which was filed on Jan. 14, 2022 and is incorporated herein by reference in its entirety.

### FIELD OF TECHNOLOGY

The present disclosure relates generally to ultraviolet (UV) light emitting diode (LED) technology and, more specifically, to an apparatus and method for air purification and sterilization in air conditioner systems and/or air ventilation systems.

### BACKGROUND

Air purification for air conditioners and/or air ventilation systems can be important to eliminate viruses, bacteria, and/or other hazardous micro-organisms from the air flowed through these systems. One such technique makes use of Ultraviolet (UV) light. UV is a form of electromagnetic radiation with wavelength between 100 nm and 400 nm, shorter than that of visible light, but longer than X-rays. UV radiation—which is divided into three bands: UVA (315-400 nm), UVB (280-315 nm), and UVC (200-280 nm), VUV (100-200 nm) is present in sunlight, and constitutes about 10% of the total electromagnetic radiation output from the Sun. UV light interacts with matter in a variety of ways. For example, short-wave UV light (e.g., UVC light) deactivates the DNA and RNA of microorganisms like bacteria, viruses, and other pathogens, and disrupts their ability to multiply and cause diseases. Due to this effect, UVC light can be used to quickly (e.g., within minutes) sterilize objects, large surfaces, or even the air in hospitals, medical centers, food plants, office spaces, etc. Advantageously, the UVC treatment leaves no residue, and thus, the treated object or area can be immediately used after sterilization. The UVC light used in sterilization applications has a wavelength between 200 and 280 nanometers, and more preferably a wavelength of 253.7 nm.

Incorporating a UV sterilization system into a conventional air conditioning and/or air ventilation system can be challenging, as many air conditioning and/or air ventilation systems vary in configuration. Conventional UV sterilization systems make use of custom configurations to fit individual air conditioning and/or air ventilation systems, which can be complicated to design, difficult to install, and costly to manufacture.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY

A UV sterilization system is disclosed. In one embodiment, the UV sterilization system includes a first UV LED device having a LED circuit and LED emitters. The UV sterilization system can include a sensor electrically coupled to the first UV LED device, the sensor configured to interrupt the operation of the first UV LED device upon detecting movement within an air conditioning system. The UV sterilization system can include a power module electrically coupled to the sensor, where the power module is electrically coupled to a power supply of the air conditioning system. In some embodiments, the air conditioning system can include a packaged terminal air conditioner (PTAC) unit or a split-air air-conditioner system, and the UV sterilization system can be a part of the PTAC unit or split-air air-conditioning system. In some examples, the sensor can include a motion sensor and/or an occupancy sensor.

A UV sterilization system is disclosed. In one embodiment, the UV sterilization system can include a plurality of UV LED devices serially coupled together, each of the UV LED devices comprising a LED circuit and LED emitters. The UV sterilization system can include a PIR sensor electrically coupled to the plurality of UV LED devices, the PIR sensor configured to interrupt the operation of the plurality UV LED devices upon detecting movement within an air conditioning system. The UV sterilization system can include a power module electrically coupled to the PIR sensor, wherein the power module is electrically coupled to a power supply of the air conditioning system.

The above and other preferred features, including various novel details of implementation and combination of events, will now be more particularly described with reference to the accompanying figures and pointed out in the claims. It will be understood that the particular systems and methods described herein are shown by way of illustration only and not as limitations. As will be understood by those skilled in the art, the principles and features described herein may be employed in various and numerous embodiments without departing from the scope of any of the present inventions. As can be appreciated from the foregoing and the following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

The foregoing Summary, including the description of some embodiments, motivations therefor, and/or advantages thereof, is intended to assist the reader in understanding the present disclosure, and does not in any way limit the scope of any of the claims.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1A is an illustration of a UV sterilization system, in accordance with some embodiments.

FIG. 1B is an illustration of a UV sterilization system, in accordance with some embodiments.

FIG. 2 is an illustration of a UV sterilization system, in accordance with some embodiments.

FIG. 3 is an illustration of a UV sterilization system, in accordance with some embodiments.

FIG. 4 is an illustration of a UV sterilization system, in accordance with some embodiments.

FIG. 5 is an illustration of a UV sterilization system, in accordance with some embodiments.

FIG. 6 is an illustration of a UV LED device with a two-circuit configuration, in accordance with some embodiments.

FIG. 7 is an illustration of a UV LED device with a tree-circuit configuration, in accordance with some embodiments.

FIG. 8 is an illustration of a UV LED device with a two-circuit configuration, in accordance with some embodiments.

FIG. 9 is an illustration of a UV LED device with a tree-circuit configuration, in accordance with some embodiments.

FIG. 10 is an illustration of a UV LED device with a two-circuit configuration, in accordance with some embodiments.

FIG. 11 is an illustration of a UV LED device with a tree-circuit configuration, in accordance with some embodiments.

FIG. 12 is an illustration of a UV LED device with a n-circuit configuration, in accordance with some embodiments.

FIG. 13 is a block diagram of an example computer system, in accordance with some embodiments.

FIG. 14 is an illustration of a UV LED device with a two-circuit configuration on separate LED boards, in accordance with some embodiments.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

Apparatus and methods for air purification and sterilization in air conditioner systems and/or air ventilation systems are presented. It will be appreciated that, for simplicity and clarity of illustration, where considered appropriate, reference numerals may be repeated among the figures to indicate corresponding or analogous elements. In addition, numerous specific details are set forth in order to provide a thorough understanding of the exemplary embodiments described herein. However, it will be understood by those of ordinary skill in the art that the exemplary embodiments described herein may be practiced without these specific details.

It can be important to eliminate viruses, bacteria, and/or other hazardous micro-organisms from air flow through an air conditioning and/or air ventilation system. This is to ensure the health and safety of individuals that receive air from these systems. Air purification through UV sterilization can be used to eliminate such viruses, bacteria, and/or other hazardous micro-organisms. Thus, it can be valuable to efficiently and effectively incorporate UV sterilization systems into conventional air conditioning and/or air ventilation systems, including Packaged Terminal Air Conditioner (PTAC) units, split-air air-conditioning systems, among other systems. Conventional UV sterilization systems can make use of custom mechanical configurations to fit individual air conditioning and/or air ventilation systems. Such custom mechanical configurations can be bulky, difficult to install and costly. Embodiments presented herein include UV sterilization systems that can eliminate and/or reduce the complexity of installing UV sterilization systems into PTAC units and split-air air-conditioning systems, and extend the lifespan the UV emitter system used as compared to other UV sterilization systems.

As used herein, the UV LEDs will be described in the context of LEDs emitting in the UVB and UVC spectrum. However, this is not limiting, and the configurations presented herein are applicable to other types of LEDs, including LEDs emitting in the UVA or visible spectrum. By way of example and not limitation, the UV LED devices will be described in the context of a system used in PTAC units or split-air air-conditioning systems for sterilization purposes—e.g., for removing pathogens from the air circulated within a room or within an enclosure. However, this is not limiting, and the configurations of the UV LED devices described herein can be standalone air sanitizing/purifying units or units integrated to other types of ventilation systems such as in locomotives, airplanes, cars, ships, and the like. Further, the configurations of the LED devices described herein can be non-sterilizing devices, such as devices generating visible light.

### UV Sterilization System for Air Purification

A UV sterilization system for air purification is presented. In some embodiments, the UV sterilization system can be configured to be placed within and/or attached to any conventional air conditioner and/or air ventilation system. In some examples, the UV sterilization system can be configured to be used within PTAC unit, split-air air-conditioning systems, among other air conditioning, air ventilation, heating, and/or cooling units. The UV sterilization system can include a power module, a sensor, mounting components, electrical wires, electrical connectors, mechanical connectors, among other components. In some embodiments, the sensor can include a motion sensor, an occupancy sensor and/or a passive infrared sensor (PIR sensor). The sensor can be configured to automatically turn-off and/or turn-on power for the UV sterilization system, e.g., the sensor can be used to prevent the accidental irradiation of components, personal and/or the UV sterilization system itself during operation. In one example, the sensor can be used in place of a mechanical switch. The mounting components can include double-sided tape, screws, brackets, magnets, neodymium rare earth magnets, among other components. The mounting components can also be referred to as interlocking components, among other terms. The electrical wires, electrical connectors, and/or mechanical connectors can include heat resistant materials, fire retardant materials, smoke limiting and/or resistant materials, among other materials. The UV sterilization system can include a modular configuration, as further described below.

In some embodiments, the modular configuration of the UV sterilization system can be configured to allow the UV sterilization system to fit within, and/or attach to a PTAC unit, split-air air-conditioning system, among any other conventional air conditioner and/or air ventilation system. The modular configuration can allow for and/or include replaceable and/or reconfigurable components. The modular configuration can allow the UV sterilization system to be placed within an air conditioner and/or air ventilation system installed at, within and/or as part of a room, a house, a building, a commercial site (e.g., a mall, a hospital, among other commercial sites), an industrial building (e.g., a manufacturing site, among other industrial sites), a car, a train, a boat, an airplane, a train station, an airport, among other locations.

In some embodiments, the UV sterilization system can include an optional multi-circuit, automatic advancing cycling and/or alternating module that can be configured to move the UV sterilization system's power from one UV LED device, e.g., which includes a UV LED circuit, to the next UV LED device every time power to the UV sterilization system is cycled to turn off and powering the next UV LED device when power is switched to turn on. In some examples, powering subsequent UV LED devices can include powering a UV LED device A, followed by powering a UV LED Device B, and followed by powering a UV LED device C. A redundancy of UV LED emitters per module and/or, a redundancy of UV LED devices and/or UV LED modules can be used. In a particular non limiting example, if a UV sterilization system uses double the lifespan of existing UV LED device, it may benefit from using one or more UV LED devices and/or using a dual circuit UV sterilization system. In some embodiments, a UV LED device, which can also be referred to as a UV LED module, having a set of UV LED circuits configured to produce and/or expose light at a target UV power output can be used. A second circuit on the UV LED device having a second set of UV LED circuits of the same output as the LEDs of the first UV LED device, or identical to the first UV LED device, can also be used. The UV LED device can be understood be to include a UV LED circuit ‘A’, while the second circuit of the multi-circuit UV LED device can be understood to include a UV LED circuit ‘B’. The UV sterilization system of the UV LED device can be installed within PTAC, split-air air-conditioning system, among other systems, and every time the air conditioning system is activated the UV sterilization system can move the UV LED device and/or UV LED circuit from one device/circuit to the next. The UV sterilization system can be configured to switch power from the UV LED circuit ‘A’ and provide the power to UV LED circuit ‘B’, and if there are additional UV LED circuits, the UV sterilization system can cycle from one to the next UV LED device and/or UV LED circuit. In some examples, cycling through UV LED circuits and/or UV LED devices can divide the hours between UV emitters used, increasing the lifespan of any UV LED device and UV sterilization system as a whole. In this way, adding more UV LED devices and/or UV LED circuits can further increase the lifespan of the UV sterilization system. The UV sterilization system can include a multi-circuit, automatic advancing cycling and/or alternating module to accommodate for the added UV LED devices and/or UV LED circuits used.

Exemplary UV sterilization systems that can be configured to eliminate and/or reduce the complexity of installing UV sterilization systems into a PTAC unit, split-air air-conditioning system, among any other conventional air conditioner and/or air ventilation system, and extend the lifespan of such UV sterilization systems, are presented below.

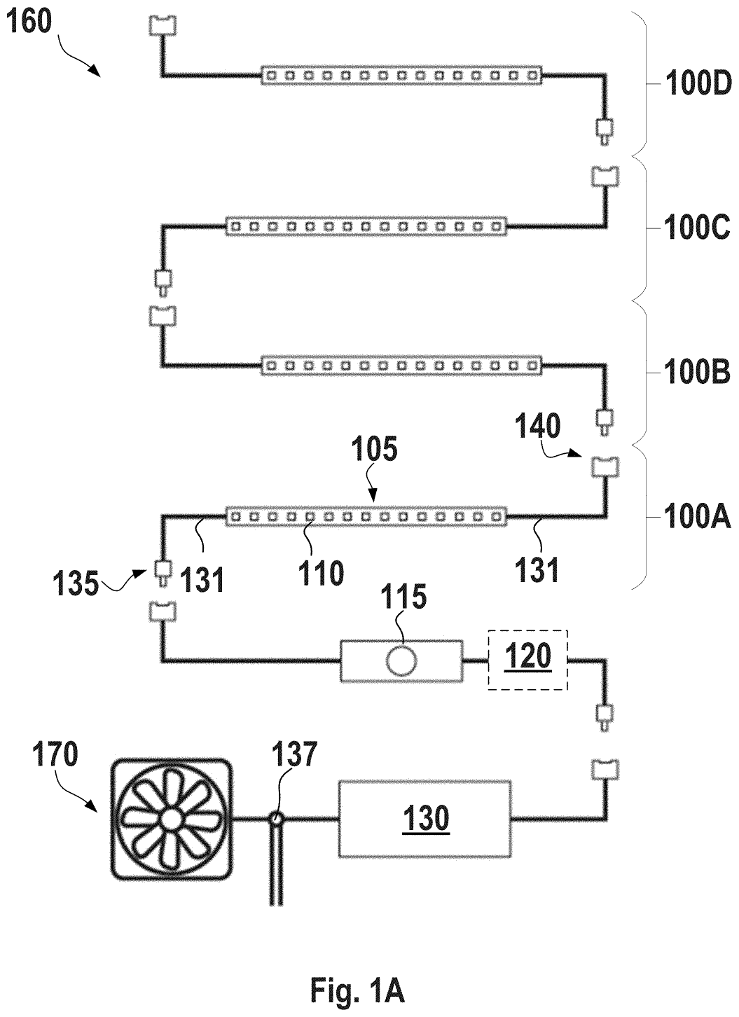

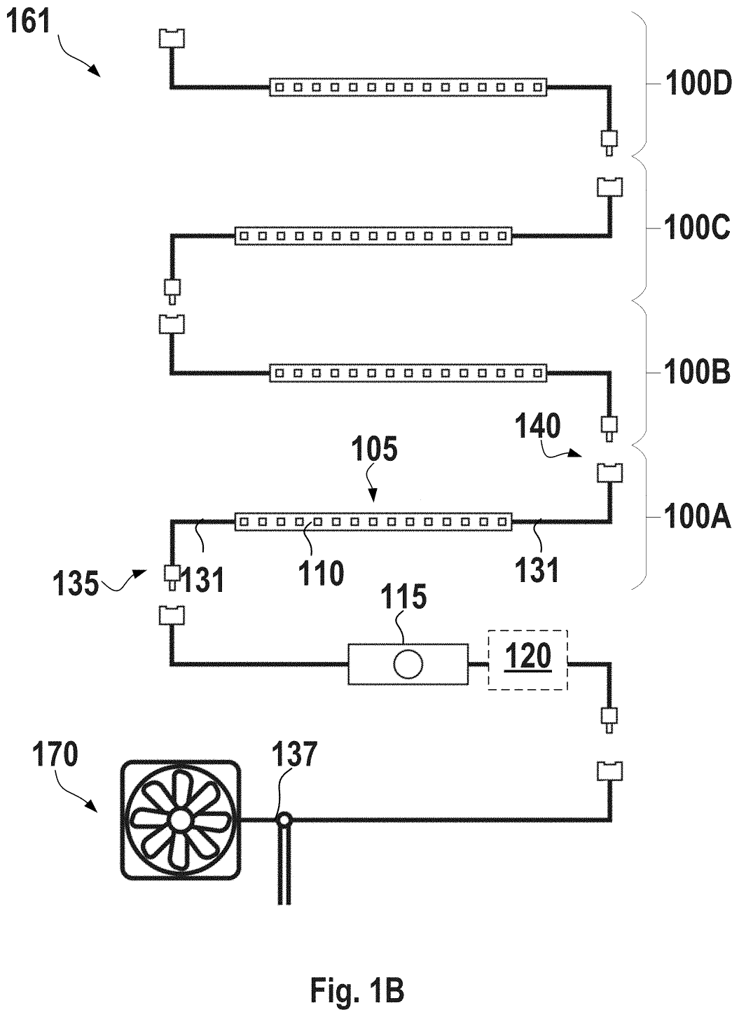

Referring to FIG. 1A, a UV sterilization system is shown according to some embodiments. The UV sterilization system 160 can include a first UV LED device 100A. The first UV LED device 100A can include a UV LED board 105 having UV LED emitters 110. The UV LED board 105 can include a printed circuitry board (PCB) or a metal core PCB (MCPCB) on which LED emitters 110 can be mechanically attached and electrically connected to. It is to be understood that UV LED board 105 can include any necessary electrical connections for the operation of LED emitters 110. Further, the UV LED board 105 can be configured so that the LED emitters 110 can be activated independently. In some embodiments, the UV LED board 105 can include a power module, e.g., the power module 130 described below. In some examples, the power module can be integrated into the UV LED board 105, can be part of the UV LED board 105, and/or attached to the UV LED board 105, e.g., each of, and/or at least one of the UV LED devices 100A-100D can include the power module. The first UV LED device 100A can include connections 135, 140. The first UV LED device 100A can include a first connection 135 and a second connection 140, as shown. The connections 135, 140 can be configured to connect the first UV LED device 100A to other electronic components. In some examples, the connections 135, 140 can be configured to connect the first UV LED device 100A to a sensor 115, a power module 130, and/or another UV LED device, among other devices and/or components. In one example, the second connection 140 can be used to connect the first UV LED device 100A to a second UV LED device 100B. As described herein, the connections 135, 140 can also be referred to as connections, electrical connections, and/or mechanical connections, among other terms. As referred to herein, the connections 135, 140 can be collectively referred to as connections and/or connectors. The first UV LED device 100A can include electronic wires 131. The electronic wires 131 can include heat resistant materials, fire retardant materials, smoke limiting and/or resistant materials, among other materials.

Referring again to FIG. 1A, the UV sterilization system 160 can include additional UV LED devices: the second UV LED device 100B, a third UV LED device 100C and a fourth UV LED device 100D. The second UV LED device 100B can be connected to the first UV LED device 100A via the connection 140, and each succeeding UV LED device, e.g., the third and fourth UV LED devices 100C, 100D, can be connected in a similar way to its adjacent UV LED device as shown. In some examples, the UV LED devices 100A-100D can be connected electrically in series and/or in parallel. The connections 135, 140 can be configured to allow for series and/or parallel connection between the UV LED devices 100A-100D. In one embodiment, the UV sterilization system 160 can include four UV LED devices 100A-100D. Although four UV LED devices 100A-100D are shown, one or more UV LED devices can be used. As referenced herein, UV LED device 100 can be used to refer to the UV LED devices 100A-100D collectively. In some examples, the labels shown for the first UV LED device 100A can be understood to be represent the components of other UV LED devices, e.g., the other UV LED devices 100B, 100C, and 100D.

In some embodiments, having multiple UV LED devices 100A-100D as shown in FIG. 1A, can allow for increased air purification coverage throughout a PTAC, split-air air conditioning system, among other air conditioner and/or an air ventilation systems. In some examples, using multiple UV LED devices, such as the UV LED devices 100A-100D, can increase the volume of air sterilized by the UV sterilization system, e.g., as opposed to using just a single UV LED device. Using multiple UV LED devices 100A-100D can be configured to fit into and/or be adapted to attach within any air conditioning and/or air ventilation system. In one example, the UV sterilization system 160 can be configured to be placed within an air intake and/or air exhaust vent of an air conditioning and/or air ventilation system. The UV LED devices 100A-100D can be configured to be attached within the air intake and/or air exhaust vent of any air conditioning and/or air ventilation system, e.g., regardless of the size, shape and/or location which the air intake and/or air exhaust vent is placed. In one example, a user can choose to install all four UV LED devices 100A-100D of the UV sterilization system 160 into an exhaust vent or intake of a PTAC unit or split-air air conditioning system. In another example, the user can choose to install the first and second UV LED devices 100A, 100B only, e.g., provided the air exhaust vent and/or air intake of the PTAC unit or split-air air conditioning system can only accommodate two UV LED devices.

As depicted in FIG. 1A, circuits UV LED devices 100A-100D can be arranged in a serial configuration to form a linear strip. However, this is not limiting, and the UV LED devices 100A-100D can be arranged in any desirable or suitable configuration. For example, the UV LED devices 100A-100D can form parallel rows configured in linear strips, parallel columns, concentric circles, a polygonal shape (e.g., square, rectangle, triangle, etc.), among any other configuration. These and other desirable and possible configurations are within the spirit and the scope of this disclosure.

In some embodiments, the UV LED devices 100A-100D can include mounting components. In some examples, the mounting components can include double-sided tape, screws, brackets, magnets, neodymium rare earth magnets, among other components. The mounting components can be configured to allow the UV LED devices 100A-100D to be attached within, coupled to and/or be part of a PTAC unit, split-air air conditioning system, or any air conditioning system and/or air ventilation system.

Referring again to FIG. 1A, in some embodiments, the UV sterilization system 160 can also include a sensor 115. In some examples, the sensor 115 can include a motion sensor, and/or an occupancy sensor. As described herein, the sensor 115 can also be referred to as a motion sensor and/or an occupancy sensor. The UV LED devices 100A-100D can be electrically coupled to the sensor 115 via the first connection 135. The sensor 115 can be configured to interrupt the operation of UV LED devices 100A-100D when the sensor 115 detect movement, detects the presence of a user, and/or is accidently accessed to protect the user from the emitted UV light. By way of example and not limitation, the sensor 115 can be a PIR sensor, a microwave occupancy sensor, or any suitable sensor configured to detect the presence of a person in the path of the emitted UV light. In one example, the sensor 115 can be used in place of a mechanical switch, e.g., to automatically turn-off and/or allow the UV sterilization system to turn-on after a duration when no presence of a person is detected. The sensor 115 can be configured to limit the operation of the UV sterilization system 160 for when air motion is detected to be at its maximum within a PTAC and/or split-air air conditioning system, e.g., when the PTAC and/or split-air air conditioning system is substantially in use. In the same example, the sensor 115 can include an airflow sensor, an air pressure sensor, among other sensors configured to detect the flow of air within an air conditioner and/or air ventilation system. The sensor 115 can be electrically coupled to a power module 130.

In some embodiments, the power module 130 can be configured to provide power to the sensor 115 and the UV LED devices 100A-100D. In some examples, power module 130 can be configured to provide power from a PTAC unit, split-air air-conditioner, or any air conditioner and/or air ventilation system. In one particular non limiting example, a connection 137 can be used to electrically couple the power module 130 to a fan power circuit 170, an air conditioning system power supply, and/or a primary ‘ON/OFF’ power switch of the PTAC unit, split-air air-conditioner system, or any air conditioner system. In some examples, when the PTAC unit and/or split-air system is not operating, the power module 130 can be configured to turn OFF individual UV LED devices 100A-100D to reduce operational cost. As described herein, the power module 130 can also be referred to as a power supply 130.

Although a power module 130 is shown external to the UV LED devices 100A-100D in FIG. 1A, in some embodiments, at least one of the UV LED devices 100A-100D can instead include the power module 130. In some examples, each of the UV LED devices 100A-100D can include their own power module. FIG. 1B shows an exemplary UV sterilization system 161 that can include at least one UV LED device 100A-100D having a power module. As described above, in some examples, the UV LED board 105 of a UV LED device can include the power module. In some examples, the power module 130 can instead be integrated into, be part of, and/or be attached to a UV LED board of at least one of the boards 100A-100D.

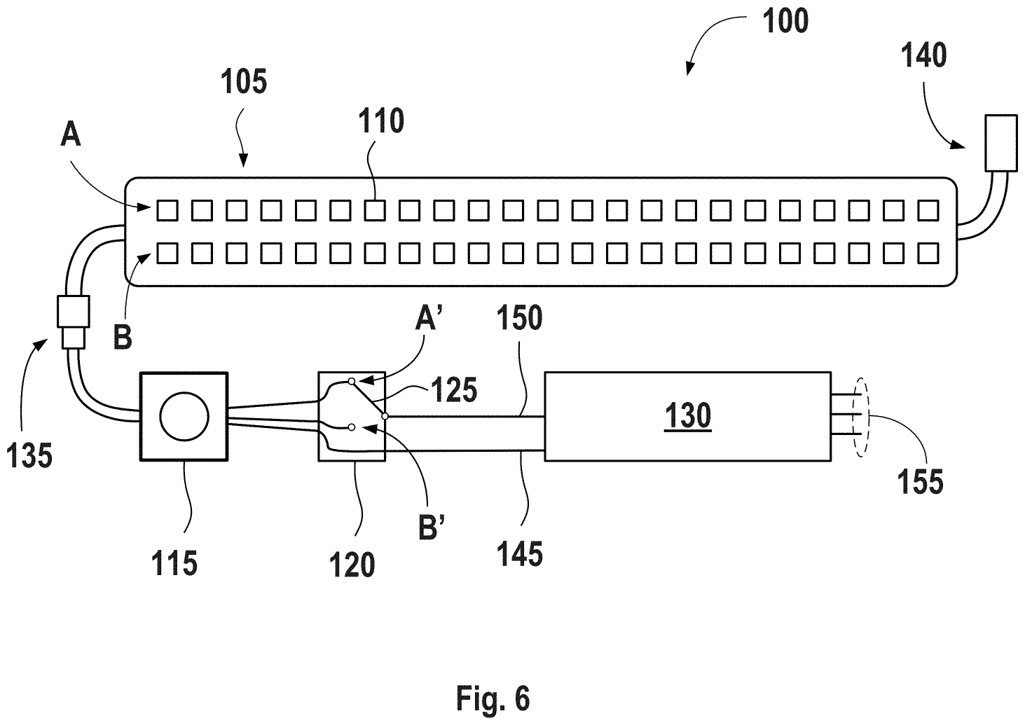

Referring to FIG. 1A, in some embodiments, the UV sterilization system 160 can include an optional automatic alternating circuit module 120. In some examples, the sensor 115 can be electrically coupled to the automatic alternating circuit module 120. The automatic alternating circuit module 120 can be configured to activate the UV LED devices 100A-100D. In some examples, the automatic alternating circuit module 120 can be configured to activate the first UV LED device 100A and, subsequently, activate the second UV LED device 100B, third UV LED device 100C, and fourth UV LED device 100D, e.g., activating one UV LED device subsequently after another. In some embodiments, the automatic alternating circuit module 120 can activate the UV LED devices 100A-100D using a selector, e.g., see selector 125 of FIG. 6. The automatic alternating circuit module 120 can be connected to the power module 130 to supply power to the UV LED devices 100A-100D. The automatic alternating circuit module 120 can use a relay, sensing, in one example, when an individual UV LED device from the UV LED devices 100A-100D is activated, and controlling when power from the power module 130 is provided to the individual UV LED devices 100A-100D. In some examples, the automatic alternating circuit module 120 can be configured to control the power module 130 OFF and then back ON. In other words, the automatic alternating circuit module 120 can cycle to the next available UV LED device each time the power from the power module 130 is switched OFF and then back ON. For example, the UV LED device 100A is activated while the power from power supply is 130 is ON, when the power is switched OFF, the automatic alternating circuit module 120 can use a selector to toggle from activating the UV LED device 100A to activating the UV LED device 100B when the power is turned back ON (e.g., see selector 125 of FIG. 6). Consequently, the LED emitters 110 in the UV LED device 100B can be activated until the next time the power from power module 130 is switched OFF and then back ON. Although not shown, connectors can be used to electrically couple the sensor 115 to the automatic alternating circuit module 120.

Alternatively, the optional automatic alternating circuit module 120 can be connected to a compressor of the PTAC or the split-air system so that every time the compressor cycles, the automatic alternating circuit module 120 can switch to the next UV LED device. In some embodiments, UV LED devices 100A-100D can be controlled independently of the PTAC or the split-air system—e.g., via a separate local or remote control unit or system. Although the PTAC and/or the split-air air conditioning system is described, the UV sterilization and the described components of the UV sterilization system, e.g., the sensor 115, power module 130, etc., can be used with any air conditioner and/or air ventilation system.

Advantageously, by performing the operation described above, the UV LED devices 100A-100D can be alternated during each power OFF/ON cycle. Thus, UV sterilization system can operate at half of the total operating time, which means that the lifetime of each of the UV LED devices 100A-100D can double, and effectively double the lifetime of the entire UV sterilization system 160.

In some embodiments, the optional automatic alternating circuit module 120 can include an air pressure switch and/or air flow sensor. In some examples, anytime an air flow or pressure change is detected, e.g., within the PTAC unit or split-air air conditioning system, power can be provided by the power module 130, and the automatic alternating circuit module 120 can cycle the power provided between the first UV LED device 100A and the second UV LED device 100B to divide the operating hours on each circuit. Thus, doubling the lifespan of the UV LED emitters in UV LED devices 100A, 100B. By way of example and not limitation, air flow or pressure changes can be caused by the operation of one or more motor fans in the PTAC or split-air air-conditioning system to which the UV LED devices 100A-100D are connected. By way of example and not limitation, when the air flow or pressure changes (e.g., increases) due to the operation of one or more motor fans 170 in the PTAC or split-air air-conditioning system, the automatic alternating circuit module 120 can be configured to activate the UV LED device 100A. When the air flow or pressure decreases (e.g., when the motor fan stops) and subsequently increases again (indicating that the motor fan is again operational), the automatic alternating circuit module 120 can be configured to activate the UV LED device 100B instead of the UV LED device 100A. The UV LED device 100A can be reactivated by the automatic alternating circuit module 120 in the next air flow or pressure change cycle as described above. As described herein the automatic alternating circuit module 120 can also be referred to as an alternating circuit module 120, among other terms.

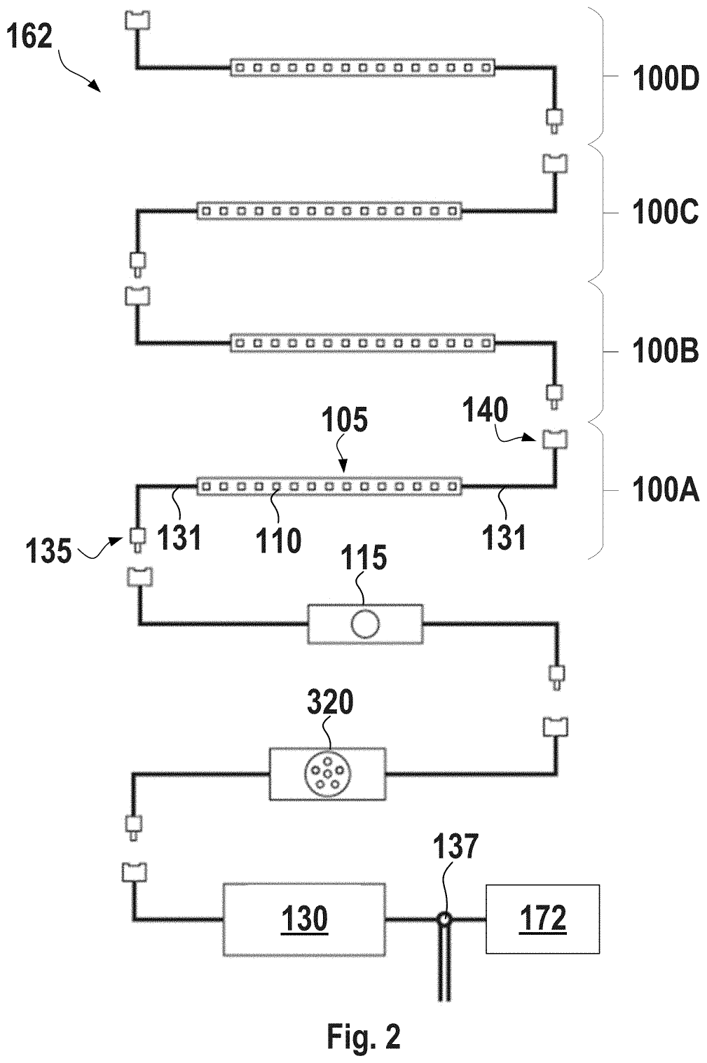

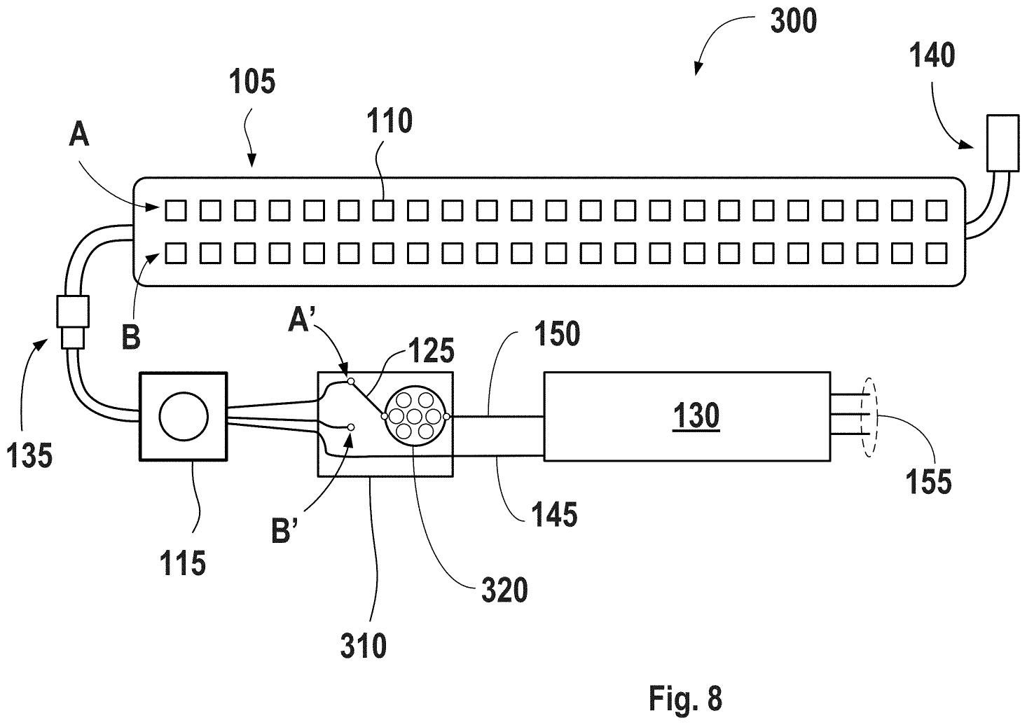

Referring to FIG. 2, a UV sterilization system 162 is shown that includes the same configuration of the UV sterilization system 160 from FIG. 1A with the exception that the automatic alternating circuit module 120 is replaced with an air flow sensor module 320 and that the fan power circuit 170 is replaced with an air conditioning system power supply 172. In some examples, the air flow sensor module 320 can include a DC Low voltage air flow sensor switch module. In one example, the air flow sensor module 320 can include the air flow sensor module 320 described in FIG. 8.

Referring to FIG. 1B, a UV sterilization system 161 is shown that includes the same configuration of the UV sterilization system 160 from FIG. 1 with the exception that at least one of, and/or each of, the UV LED devices 100A-100D can include a power module. In some examples, at least one of, and/or each of, the UV LED devices 100A-100D can include the power module to be used by the UV sterilization system 161. In some embodiments, the UV sterilization system 161 includes an optional automatic alternating circuit module 120 as shown, where other embodiments of the UV sterilization system 161 may not include the automatic alternating circuit module 120.

Referring again to FIG. 2, in some embodiments, the air flow sensor module 320 can include an air pressure switch and/or air flow sensor. In some examples, anytime an air flow or pressure change is detected, e.g., within the PTAC unit or split-air air conditioning system, power can be provided by the power module 130, and the air flow sensor module 320 can cycle the power provided between the first UV LED device 100A and the second UV LED device 100B to divide the operating hours on each circuit. Thus, doubling the lifespan of the UV LED emitters in UV LED devices 100A, 100B. By way of example and not limitation, air flow or pressure changes can be caused by the operation of one or more motor fans, or by activation of the air conditioning system power supply 172, in the PTAC or split-air air-conditioning system to which the UV LED devices 100A-100D are connected. By way of example and not limitation, when the air flow or pressure changes (e.g., increases) due to the operation of the air conditioning system power supply 172 in the PTAC or split-air air-conditioning system, the automatic alternating circuit module 120 can be configured to activate the UV LED device 100A. When the air flow or pressure decreases (e.g., when the motor fan stops) and subsequently increases again (indicating that the motor fan is again operational), the automatic alternating circuit module 120 can be configured to activate the UV LED device 100B instead of the UV LED device 100A. The UV LED device 100A can be reactivated by the automatic alternating circuit module 120 in the next air flow or pressure change cycle as described above.

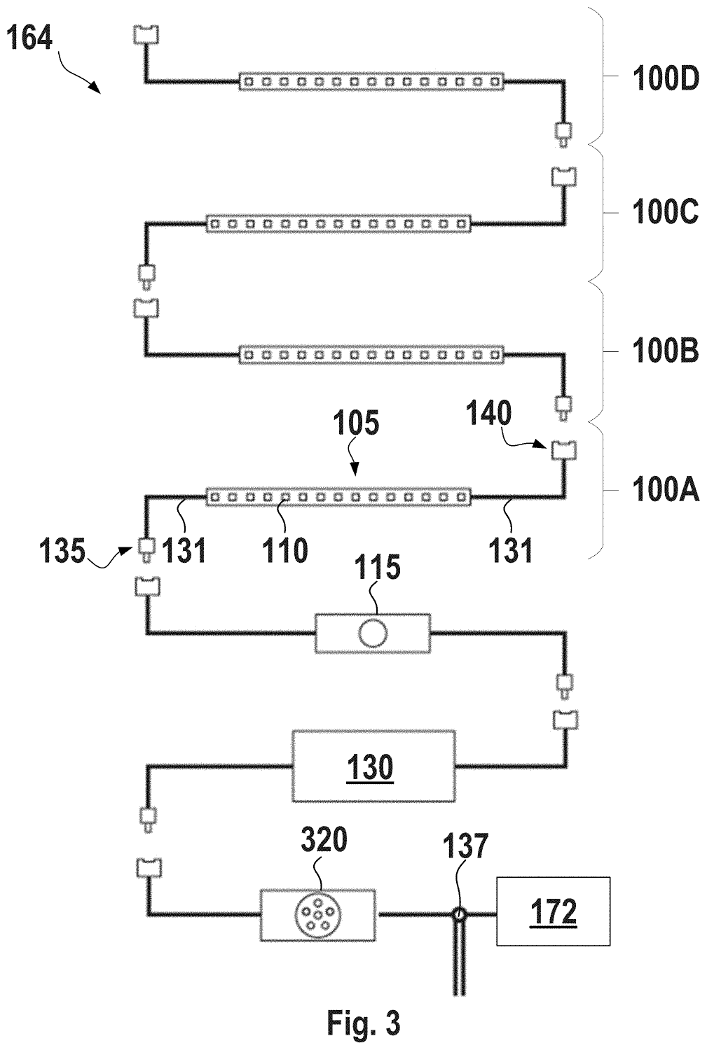

Referring to FIG. 3, a UV sterilization system 164 is shown that includes the same configuration of the UV sterilization system 162 from FIG. 2 with the exception that the air flow sensor module 320 is placed after the power module 130.

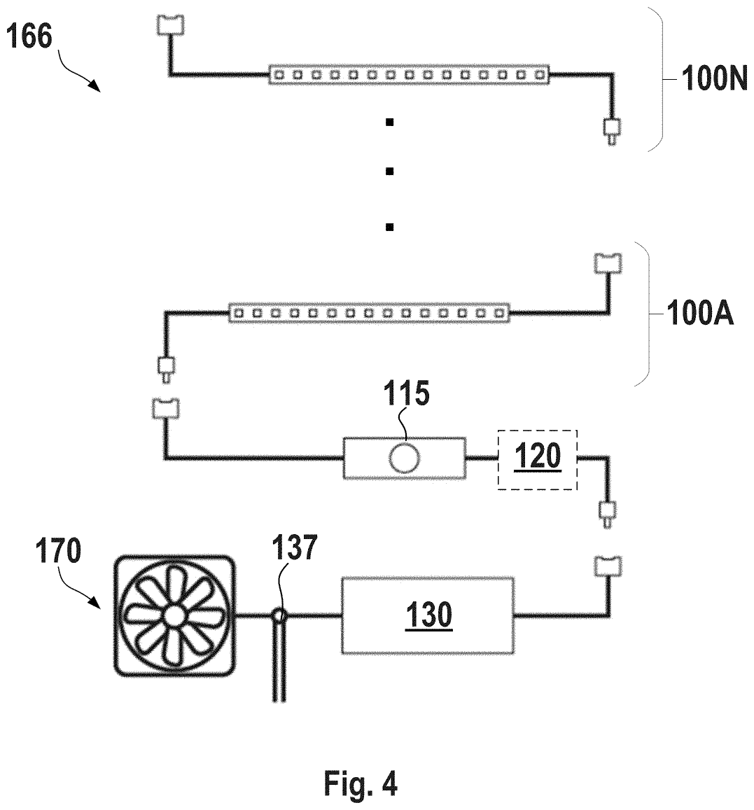

Referring to FIG. 4, a UV sterilization system 166 is shown that includes the same configuration of the UV sterilization system 160 from FIG. 1A, where instead of including 4 UV LED devices 100A-100D, the UV sterilization system 166 includes a n number UV LED devices, where n can represent any integer number of UV LED devices. In some examples, the UV sterilization system 166 can have n number UV LED devices, where the UV LED device 100N is the nth UV LED device.

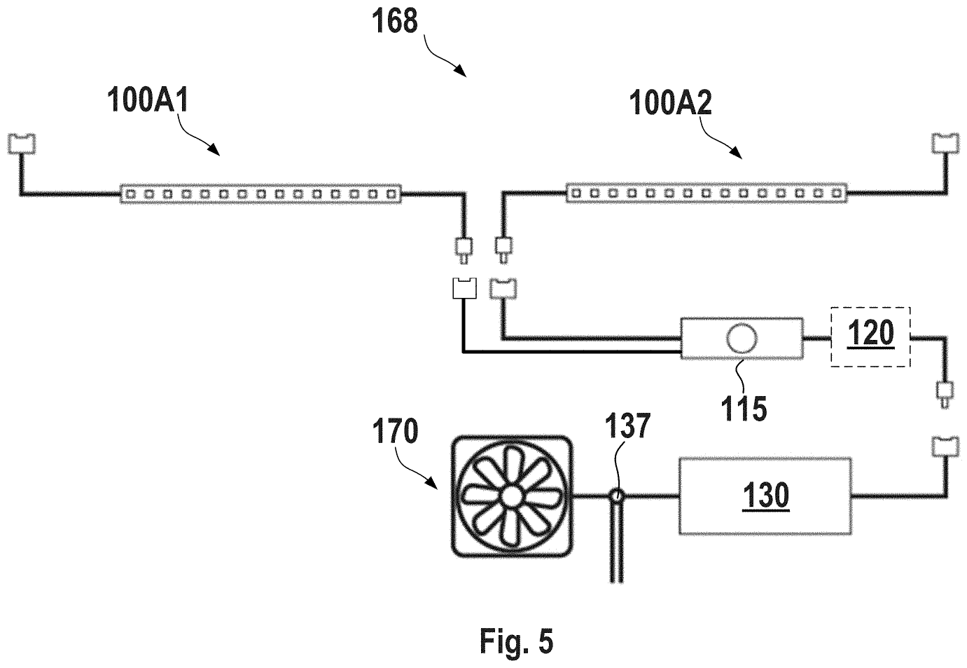

Referring to FIG. 5, a UV sterilization system 168 is shown that includes the same configuration of the UV sterilization system 160 from FIG. 1A with the exception that UV LED devices 100A1 and 100A2 are connected in a parallel configuration, e.g., as opposed to a serial configuration shown in FIG. 1A. In some examples, the UV LED devices 100A1, 100A2 can be electrically coupled to the sensor 115.

In some embodiments, the UV sterilization systems 160-168 described in FIGS. 1-5 can be configured to be modular, compact and/or portable such that the UV sterilization system can be placed within any air conditioner and/or air ventilation system. In some examples, UV sterilization systems 160-168 can be configured to be accessed, controlled and/or triggered remotely. The UV sterilization systems 160-168 can be configured to be connected to a remote access device and/or remote access electronics. The UV sterilization systems 160-168 can be wirelessly triggered and/or can be controlled wirelessly. The UV sterilization systems 160-168 can be accessed via a mobile phone, tablet and/or any other type of mobile electronic device. The UV sterilization systems 160-168 can be placed in any location PTAC, split-air air conditioner, air conditioner, and/or air ventilation system can be installed, e.g., a house, car, train, boat, airplane, train car, train station, building, room, industrial area, among other locations and/or places. The UV sterilization systems 160-168 can be triggered by the presence and/or absence of a person within an air conditioner and/or air ventilated area, e.g., a person entering and/or leaving a room. In one example, the sensor 115 of the UV sterilization systems 160-168 can be used to determine the presence and/or absence of a person within an air conditioner and/or air ventilated area.

In some embodiments, the UV sterilization systems 160-168 can be controlled via and/or connected to one or more computer systems. In some examples, the computer systems can be remote computer systems, configured to control, initiate and/or trigger the UV sterilization systems 160-168. The computer systems can include software that is configured to control the operation of the UV sterilization systems 160-168. In some embodiments, the software can include artificial intelligence (AI) software. In some examples, the UV sterilization systems 160-168 can be controlled, initiated and/or triggered by the software based on learned data. In one example, the UV sterilization systems 160-168 can be controlled, initiated and/or triggered by the average time users enter a building and/or the average time users leave the building. The UV sterilization systems 160-168 can be controlled, initiated and/or triggered by the peak usage hours of a transportation hub, e.g., an airport, train station, among others. In one particular non limiting example, the UV sterilization systems 160-168 can be controlled, initiated and/or triggered prior to departure, and/or before passengers board their train or flight, and/or in between train rides or flights.

It is to be appreciated that UV sterilization systems 160-168 may include additional electrical or electronic components necessary for its function. However, these additional electrical and electronic components are not shown in FIGS. 1-5 merely for simplicity. Such components may include, but are not limited to, controllers, relays, timers, processing units and modules, passive and active electronic devices, additional connections, and the like. These additional components are within the spirit and scope of this disclosure.

In some embodiments, the UV LED devices described in FIGS. 1-5 above can include the UV LED devices described in FIGS. 6-14 below, although other UV LED devices can be used.

### UV LED Product Automatic Lifespan Increaser

FIG. 6 is a UV LED device 100, according to some embodiments. UV LED device 100 includes a UV LED board 105 having UV LED emitters 110 configured in a two-circuit arrangement formed by circuits A and B. In some embodiments, UV LED board 105 is a printed circuitry board (PCB) or a metal core PCB (MCPCB) on which LED emitters 110 are mechanically attached and electrically connected to. It is to be understood that UV LED board includes any necessary electrical connections for the operation of LED emitters 110. Further, UV LED board 105 is configured so that the LED emitters 110 between circuits A and B can be activated independently. That is, the LED emitters 110 in circuit A can be operated independently from the emitters in circuit B. In some embodiments, only a single circuit may be operated at any given time—i.e., either A or B as discussed below.

According to some embodiments, the total number of LED emitters 110 selected for each of the circuits A and B is determined based on the power output of each emitter and the desired total power output of UV LED device 100. In other words, fewer or more LED emitters 110 may be used in each circuit depending on the power output of each LED emitter and the desired total output of the UV LED device 100.

As depicted in FIG. 6, circuits A and B are arranged as parallel rows in the form of linear strips. However, this is not limiting, and circuits A and B may be arranged in any desirable or suitable configuration. For example, circuits A and B may form concentric circles or may be arranged as parallel columns. These and other desirable and possible configurations are within the spirit and the scope of this disclosure.

UV LED board 105 is electrically coupled, via connection 135, to a sensor 115 configured to interrupt the operation of UV LED device 100 when the device is accidently accessed to protect the user from the emitted UV light. In some examples, the sensor 115 can include a safety occupancy motion sensor, a motion sensor and/or an occupancy sensor. As described herein, the sensor 115 can also be referred to herein as a safety occupancy motion sensor, a motion senor and/or an occupancy sensor. By way of example and not limitation, sensor 115 may be a passive infrared sensor (PIR sensor), a microwave occupancy sensor, a motion sensor, or any suitable sensor configured to detect the presence of a person in the path of the emitted UV light.

Sensor 115 is electrically coupled to an automatic alternating circuit module 120 configured to activate circuits A and B in UV LED board 105 via a selector 125. According to some embodiments, when selector 125 is at position A′, circuit A in UV LED board 105 is activated and, respectively, when selector 125 is at position B′, circuit B is activated. In some embodiments, selector 125 toggles with the help of a relay (not shown in FIG. 6 for simplicity) between positions A′ and B′ each time automatic alternating circuit module 120 “senses” via the relay (which can be interposed between selector 125 and the positive connection 150 of power supply 130) that the power provided via power supply 130 is switched OFF and then back ON. In other words, automatic alternating circuit module 120 cycles to the next available circuit (e.g., A or B) each time the power from power supply 130 is switched OFF and then back ON. For example, assuming that selector 125 is initially at position A′ while the power from power supply is 130 is ON, when the power is switched OFF, the automatic alternating circuit module 120 will toggle selector 125 from position A′ to position B′ when the power is turned back ON. Consequently, the LED emitters 110 in circuit B will be activated until the next time the power from power supply 130 is switched OFF and then back ON.

Advantageously, by performing the operation described above, the UV LED emitters 110 in each circuit A and B are alternating during each power OFF/ON cycle. Thus, UV LED emitters 110 in circuits A and B operate half of the total operating time, which means that the lifetime of the entire UV LED device 100 doubles.

By way of example and not limitation, selector 125 is electrically coupled, via the relay, to the positive connection 150 of the power supply 130 while sensor 115 is electrically coupled to power supply 130 via a common or negative connection 145. According to some embodiments, power supply 130 is also coupled to external power distributor and control equipment via connections 155. It is noted that UV LED board 105 may be electrically coupled to other boards or systems via optional connection 140.

It is to be appreciated that UV LED device 100 may include additional electrical or electronic components necessary for its function. However, these additional electrical and electronic components are not shown in FIG. 6 merely for simplicity. Such components may include, but are not limited to, controllers, relays, timers, processing units and modules, passive and active electronic devices, additional connections, and the like. These additional components are within the spirit and scope of this disclosure.

Further, and as discussed above, UV LED device 100 may be an integral part of a higher level system that controls the operation of UV LED device 100. By way of example and not limitation, UV LED device 100 may be electrically coupled to a fan motor so that when the PTAC or split-air system is not operating, the power of UV LED device 100 is turned OFF to reduce the operational cost. Alternatively, the automatic alternating circuit module 120 may be connected to a compressor of the PTAC or the split-air system so that every time the compressor cycles, the selector 125 may switch to the next circuit of emitters. In some embodiments, UV LED device 100 may be controlled independently of the PTAC or the split-air system—e.g., via a separate local or remote control unit or system.

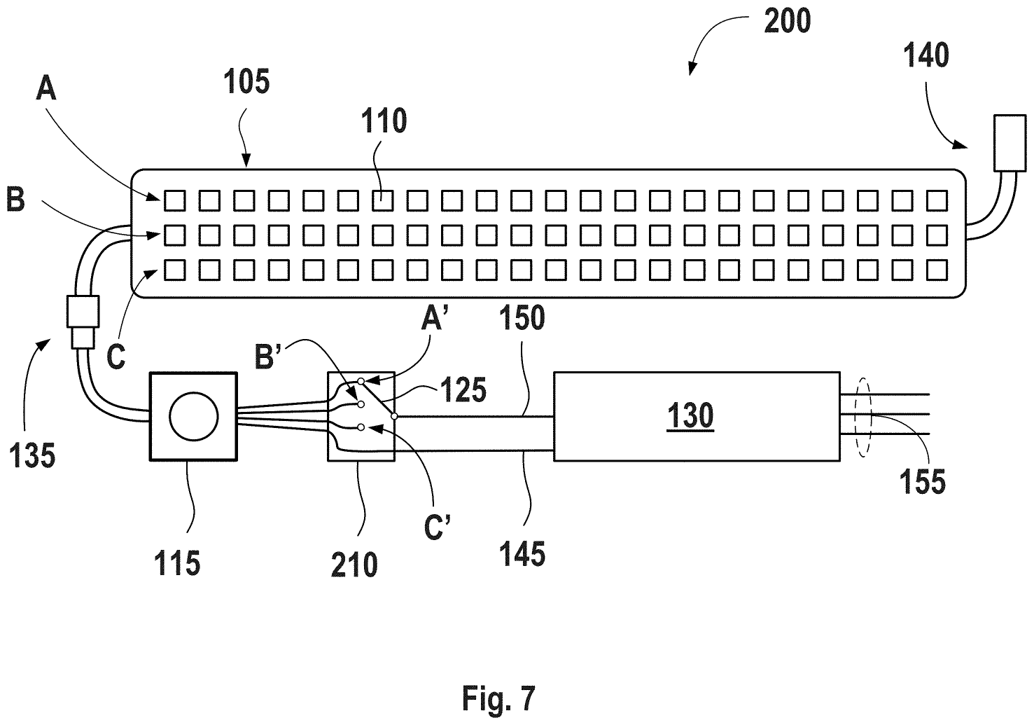

According to some embodiments, FIG. 7 shows a UV LED device 200, which is a variant of UV LED device 100 shown in FIG. 6. The difference between UV LED devices 200 and 100 is that UV LED device 200 features a three-circuit configuration as opposed to a two-circuit configuration. Further, each of circuits A, B, and C includes an equal number of UV LED emitters 110. Because UV LED device 200 has a three-circuit configuration, it also includes an automatic alternating circuit module 210 with three available positions A′, B′, and C′ for selector 125. According to some embodiments, UV LED device 200 operates in a similar fashion with UV LED device 100 with the exception that with the help of selector 125, UV LED device 200 can cycle through circuits A, B, and C for each power OFF/ON cycle. As a result, the lifetime of UV LED device 200 is further increased compared to UV LED device 100 because each circuit is operated one third of the time (e.g., ⅓) instead of half the time (e.g., ½).

In yet another embodiment, FIG. 8 shows a UV LED device 300, which has identical components to UV LED device 100 shown in FIG. 6 with the exception of automatic alternating circuit module 310 in which the position of selector 125 is controlled (via the relay) through a pressure switch or air flow sensor 320. According to some embodiments, anytime an air flow or pressure change is detected by sensor 320, power is provided by power supply 130 and selector 125 cycles between circuits A and B to divide the operating hours on each circuit. Thus, doubling the lifespan of the UV LED emitters 110 in UV LED device 300. By way of example and not limitation, air flow or pressure changes can be caused by the operation of one or more motor fans in the PTAC or split-air air-conditioning system to which the UV LED device 300 is connected. Advantageously, the position of selector 125 may toggle between positions A′ and B′ when the air flow raises above or falls below a certain threshold. By way of example and not limitation, when the air flow or pressure changes (e.g., increases) due to the operation of one or more motor fans in the PTAC or split-air air-conditioning system, selector 125 may switch to position A′ to activate circuit A. When the air flow or pressure decreases (e.g., when the motor fan stops) and subsequently increases again (indicating that the motor fan is again operational), selector 125 may switch to position B′ to activate circuit B instead of circuit A. Circuit A may be reactivated via selector 125 moving to position A′ in the next air flow or pressure change cycle as described above.

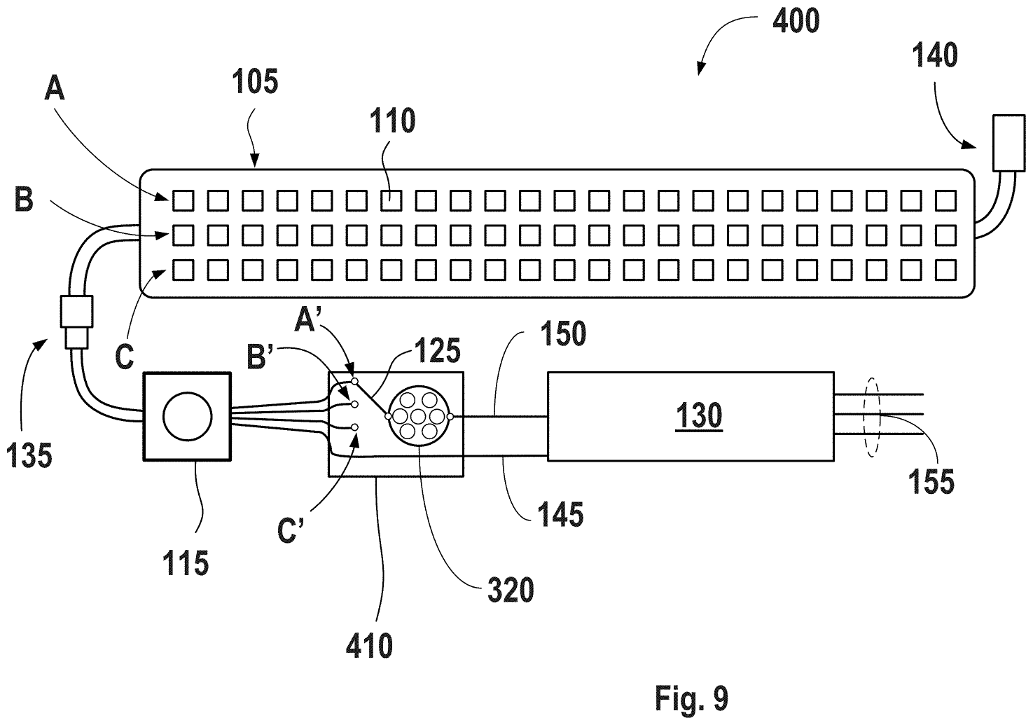

According to some embodiments, FIG. 9 shows a UV LED device 400, which is a variant of UV LED device 300, with a three-circuit system as opposed to a two-circuit system. In this configuration, UV LED device 400 is capable of operating between three LED circuits (e.g., A, B, and C) instead of just two (e.g., A and B), to further increase the lifetime of UV LED emitters 110 by cutting their operating time to one third (e.g., ⅓) from one half (e.g., ½). According to some embodiments, the operation principles of UV LED device 400 is similar to that of UV LED device 300. The only difference is that UV LED device 400 cycles through circuits A, B, and C as opposed to circuits A and B within every air flow or pressure change cycle as described above.

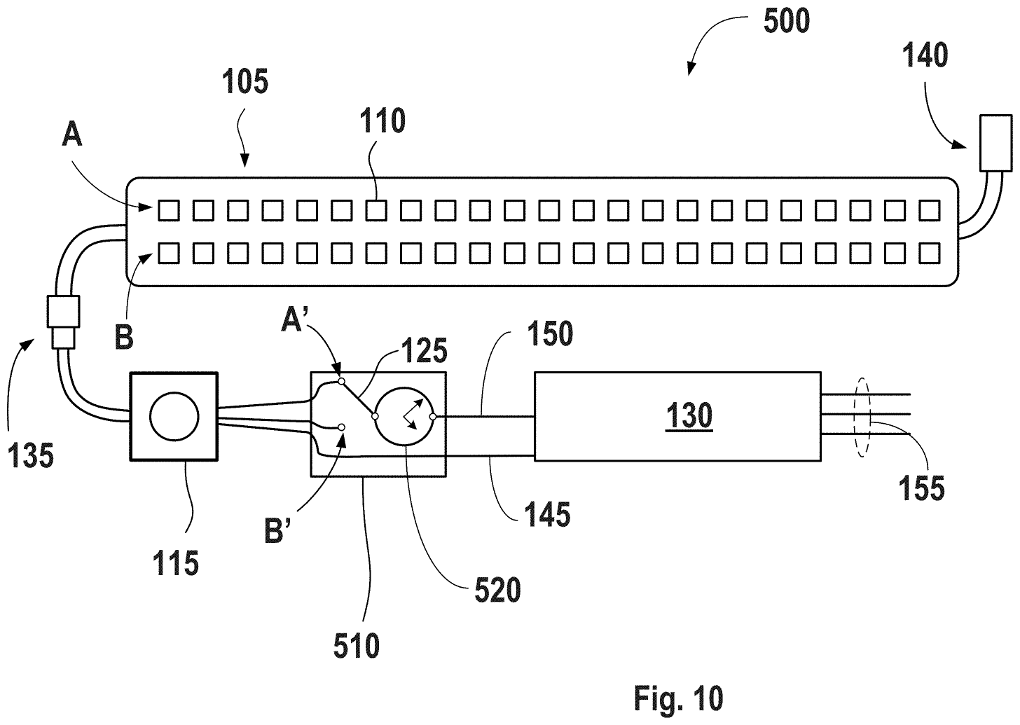

FIG. 10 shows a UV LED device 500, which has identical components to UV LED device 100 shown in FIG. 6 with the exception of an automatic alternating circuit module 510 in which the position of selector 125 between positions A′ and B′ is controlled (via the relay) through a timer 520. In this embodiment, the selector 125 activates circuits A and B based on a predetermined amount of time to ensure that circuits A and B are driven an equal amount of time during the lifetime of UV LED emitters 110. For example, circuit A may be activated for a predetermined amount of time once UV LED device 500 is powered ON. After the predetermined amount of time has elapsed, selector 125 switches to position B′ to activate circuit B either while UV LED device 500 is powered ON or the next time the UV LED device 500 is powered ON. The selector may return to position A′ once the predetermined amount of time has elapsed for a second time. The aforementioned process may repeat itself multiple times. In some embodiments, timer 520 may be programmed to any interval based on the lifetime of UV LED emitters 110 and the number of LED circuits on UV LED board of the UV LED device.

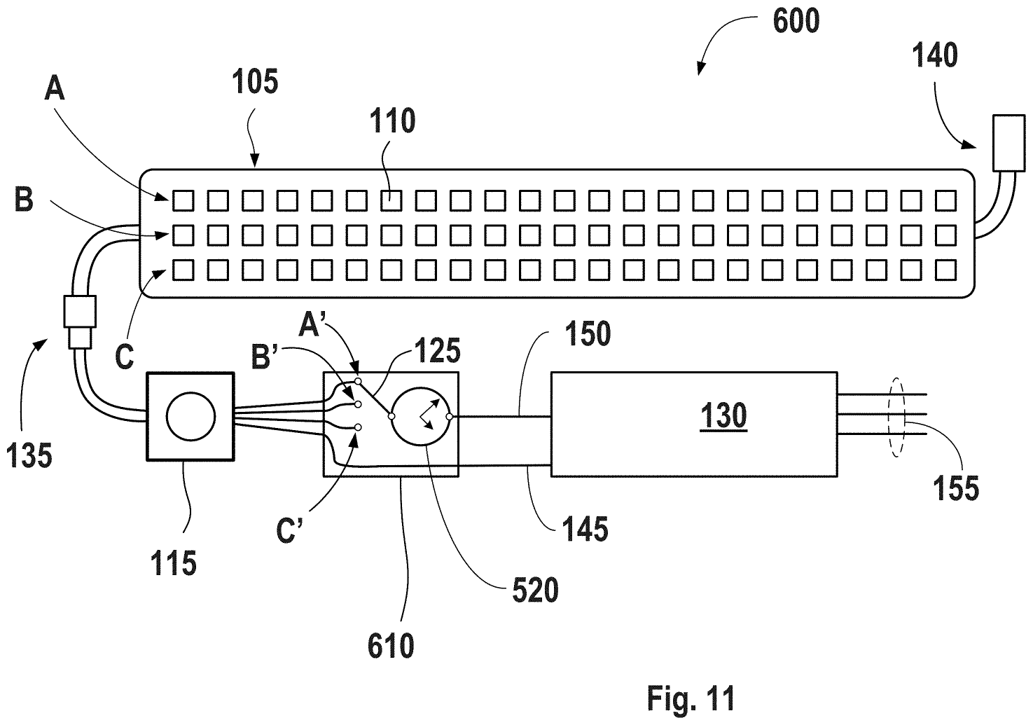

Similarly to the UV LED devices discussed above in FIGS. 1 and 3, UV LED device 500 may be equipped with a greater number of LED circuits, as shown for example in FIG. 11 for UV LED device 600. The operating principle for UV LED device 600 is similar to that for UV LED device 500 discussed above with the exception that UV LED device 600, via automatic alternating circuit module 610 and timer 520, is now configured to switch between three LED circuits (A, B, and C) instead of just two (e.g., A and B). Advantageously, UV LED device 600 reduces the operation of UV LED emitters 110 to one third (e.g., ⅓) from one half (e.g., ½), effectively increasing further its total lifetime compared to UV LED device 500, which only utilizes LED circuits A and B.

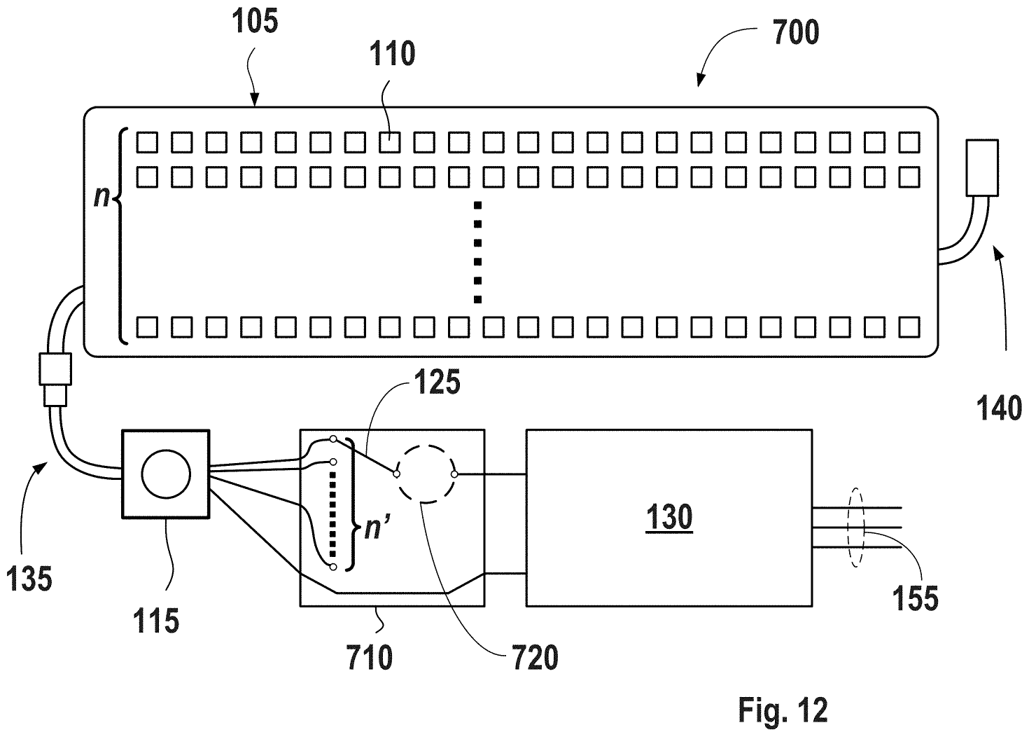

As discussed above, the UV LED devices presented herein may include a greater number of LED circuits with UV LED emitters 110 on their UV LED board 105 to further increase the lifetime of the UV LED device. For example, FIG. 12 shows such UV LED device (e.g., UV LED device 700) which includes n number of LED circuits. According to some embodiments, n is an integer equal to or greater than 2 (e.g., n>2). Accordingly, UV LED device 700 is equipped with an automatic alternating circuit module 710 featuring a selector 125 which can move between n′ positions to select any of the corresponding n LED circuits. According to some embodiments, each of the n′ position corresponds to one of the n LED circuits; thus, n′ is equal to n.

According to some embodiments, automatic alternating circuit module 710 may include a relay 720 that controls the position of connector 125. In some embodiments, relay 720 may incorporate a pressure switch or a flow sensor, like pressure switch or flow sensor 320 discussed above in connection to UV LED devices 300 and 400. In some embodiments, relay 720 may incorporate a timer, like timer 520 discussed above in connection to UV LED devices 500 and 600. In yet another embodiment, relay 720 may detect whether power is provided by power supply 130 and accordingly changing the position of connector 125 as discussed above in connection to the operation of UV LED devices 100 and 200. In some embodiments, relay 720 may incorporate any combination of the aforementioned components (e.g., timers, pressure switches, or a flow sensors). In some embodiments, power supply 130 may be optional if the power provided by the PTAC unit or split-air air-conditioning system does not require conversion or transformation.

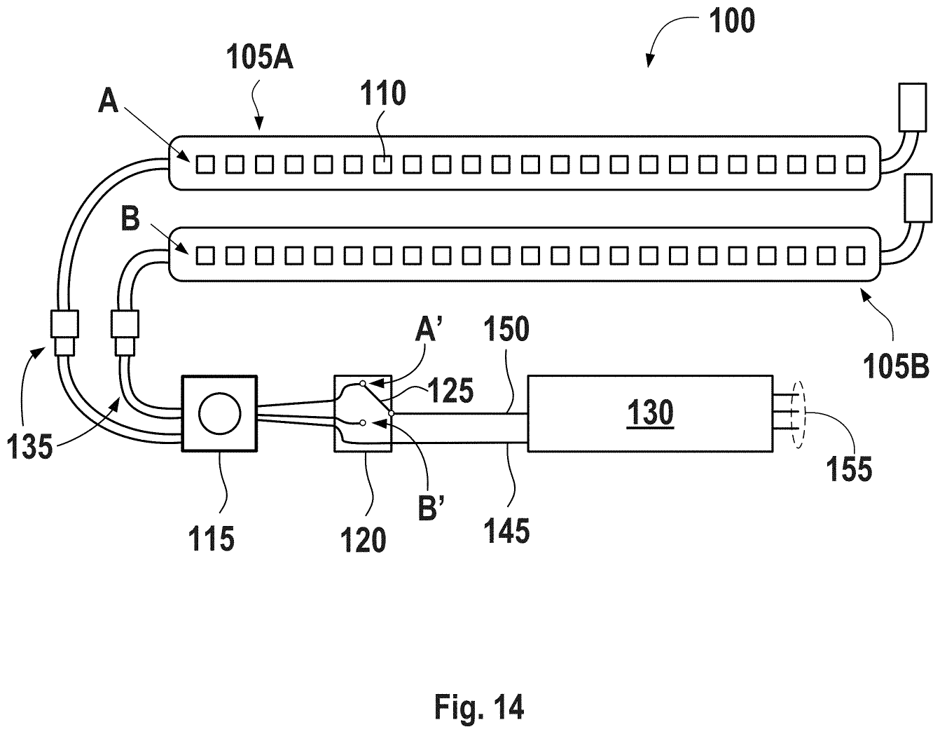

In some embodiments, instead of forming n circuits on a single UV LED board 105, the n circuits may be distributed among respective LED boards that are electrically connected to the sensor 115 via multiple connections 135. By way of example and not limitation, FIG. 14 shows the UV LED device 100 from FIG. 6 in a configuration where circuits A and B are formed on different UV LED boards 105A and 105B according to the above description.

### Software and Hardware Implementations

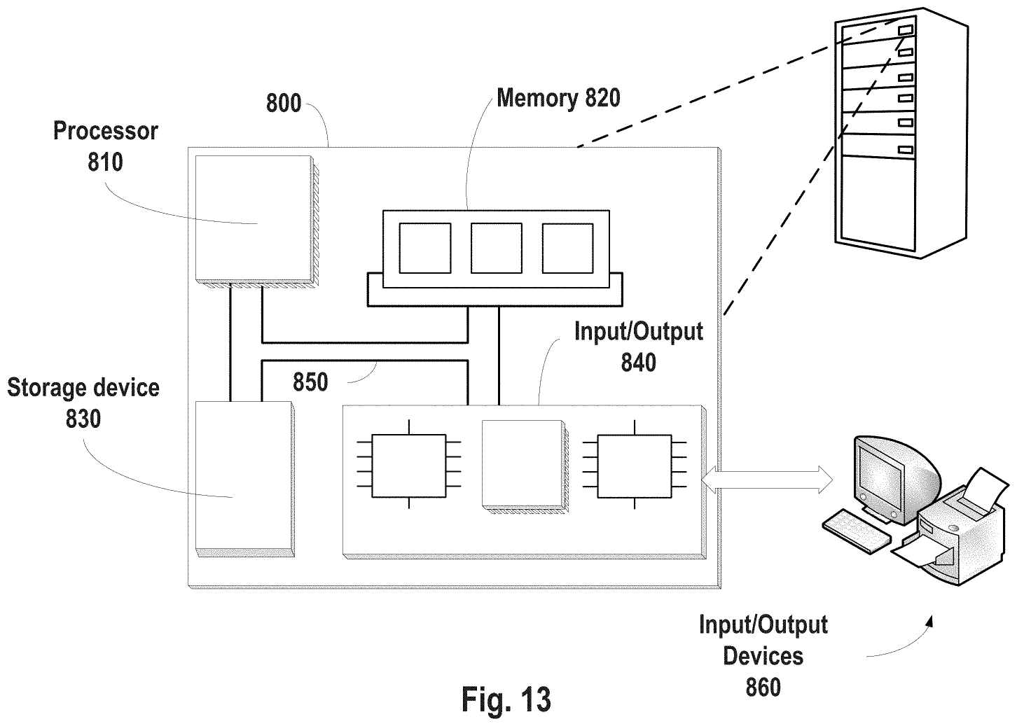

FIG. 13 is a block diagram of an example computer system 800 that may be used in implementing the technology described in this document. General-purpose computers, network appliances, mobile devices, or other electronic systems may also include at least portions of system 800. System 800 includes a processor 810, a memory 820, a storage device 830, and an input/output device 840. Each of components 810, 820, 830, and 840 may be interconnected, for example, using a system bus 850. Processor 810 is capable of processing instructions for execution within system 800. In some implementations, processor 810 is a single-threaded processor. In some implementations, processor 810 is a multi-threaded processor. In some implementations, processor 810 is a programmable (or reprogrammable) general purpose microprocessor or microcontroller. Processor 810 is capable of processing instructions stored in memory 820 or on storage device 830.

Memory 820 stores information within system 800. In some implementations, memory 820 is a non-transitory computer-readable medium. In some implementations, memory 820 is a volatile memory unit. In some implementations, memory 820 is a non-volatile memory unit.

Storage device 830 is capable of providing mass storage for system 800. In some implementations, storage device 830 is a non-transitory computer-readable medium. In various different implementations, storage device 830 may include, for example, a hard disk device, an optical disk device, a solid-date drive, a flash drive, or some other large capacity storage device. For example, the storage device may store long-term data (e.g., database data, file system data, etc.). The input/output device 840 provides input/output operations for the system 800. In some implementations, the input/output device 840 may include one or more of a network interface device, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 860. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. Storage device 830 may be implemented in a distributed way over a network, for example as a server farm or a set of widely distributed servers, or may be implemented in a single computing device.

Although an example processing system has been described in FIG. 13, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, a data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array), an ASIC (application specific integrated circuit), or a programmable general purpose microprocessor or microcontroller. A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array), an ASIC (application specific integrated circuit), or a programmable general purpose microprocessor or microcontroller.

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic disks, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

### Terminology

The phrasing and terminology used herein is for the purpose of description and should not be regarded as limiting.

Measurements, sizes, amounts, and the like may be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

Furthermore, connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

The use of certain terms in various places in the specification is for illustration purposes only and should not be construed as limiting. A service, function, or resource is not limited to a single service, function, or resource; usage of these terms may refer to a grouping of related services, functions, or resources, which may be distributed or aggregated.

Furthermore, one skilled in the art shall recognize that: (1) certain steps may optionally be performed; (2) steps may not be limited to the specific order set forth herein; (3) certain steps may be performed in different orders; and (4) certain steps may be performed simultaneously or concurrently.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A UV sterilization system, comprising:
a first UV LED device comprising a LED circuit and LED emitters;
a sensor electrically coupled to the first UV LED device, the sensor configured to interrupt the operation of the first UV LED device upon detecting movement within an air conditioning system; and
a power module electrically coupled to the sensor, wherein the power module is electrically coupled to a power supply of the air conditioning system.

2. The UV sterilization system of claim 1, wherein the sensor comprises at least one of a motion sensor, a passive infrared (PIR) sensor, an occupancy sensor, a microwave occupancy sensor, an airflow sensor, or an air pressure sensor.

3. The UV sterilization system of claim 1, wherein the power module is integrated into the LED circuit of the first UV LED device.

4. The UV sterilization system of claim 1, further comprising a second UV LED device serially coupled to a first UV LED device, the first and second UV LED devices each comprising a LED circuit and LED emitters.

5. The UV sterilization system of claim 4, further comprising a third UV LED device serially coupled to the second UV LED device, the first, second, and third UV LED devices each comprising a LED circuit and LED emitters.

6. The UV sterilization system of claim 4, wherein the first UV LED device is electrically coupled to the second UV LED device in series.

7. The UV sterilization system of claim 4, wherein the first UV LED device is electrically coupled to the second UV LED device in parallel.

8. The UV sterilization system of claim 1, wherein the power module is electrically coupled to a fan power circuit of the air conditioning system.

9. The UV sterilization system of claim 1, wherein the air conditioning system comprises at least one of a packaged terminal air conditioner (PTAC) unit or a split-air air-conditioner system.

10. The UV sterilization system of claim 1, further comprising an alternating circuit module electrically coupled to the sensor and the power module.

11. The UV sterilization system of claim 10, wherein the alternating circuit module is configured to activate the first LED device.

12. The UV sterilization system of claim 10, wherein the alternating circuit module comprises at least one of air pressure switch or an air flow sensor.

13. The LED device of claim 1, wherein the LED emitters of the first and second UV LED devices when operated generate ultraviolet light with a wavelength between 100 nm and 400 nm.

14. The LED device of claim 1, wherein the UV sterilization system is a part of a PTAC unit or split-air air-conditioning system.

15. A UV sterilization system, comprising:
a plurality of UV LED devices serially coupled together, each of the UV LED devices comprising a LED circuit and LED emitters;
a PIR sensor electrically coupled to the plurality of UV LED devices, the PIR sensor configured to interrupt the operation of the plurality UV LED devices upon detecting movement within an air conditioning system; and
a power module electrically coupled to the PIR sensor, wherein the power module is electrically coupled to a power supply of the air conditioning system.

16. The UV sterilization system of claim 15, wherein the plurality of UV LED devices are electrically coupled together in series or in parallel.

17. The UV sterilization system of claim 15, wherein the power module is electrically coupled to a fan power circuit of the air conditioning system.

18. The UV sterilization system of claim 15, wherein the air conditioning system comprises at least one of a PTAC unit or a split-air air-conditioner system.

19. The LED device of claim 15, wherein the UV sterilization system is a part of a PTAC unit or split-air air-conditioning system.

20. The UV sterilization system of claim 15, further comprising an alternating circuit module electrically coupled to the PIR sensor and the power module.
